(19) United States
(12) Patent Application Publication
(10) Pub. No.: US 2009/0259252 A1
(43) Pub. Date: Oct. 15, 2009

(54) Apparatus For The Joining Of Tissue Having Integral Penetrating End

(76) Inventors: John J. Kennedy, Guilford, CT (US); Joshua Stopek, Yalesville, CT (US)

Correspondence Address: Tyco Healthcare Group LP, 60 MIDDLETOWN AVENUE, NORTH HAVEN, CT, 06473, US

(21) Appl. No.: 12/412,613

(22) Filed: Mar. 27, 2009

Related U.S. Application Data
(60) Provisional application No. 61/044,968, filed on Apr. 15, 2008.

(51) Int. Cl. A61B 17/04; A61L 17/06

(52) U.S. Cl. 606/230; 606/228; 28/278

(57) ABSTRACT

The present disclosure relates to an apparatus for joining tissue in surgical applications and/or incision repair, and to methods for making the same. The apparatus includes an elongated member formed of a biocompatible material, and a rigidifying agent associated with a distal end portion of the elongated member, wherein the rigidifying agent increases the rigidity of the distal end portion such that the distal end portion is mechanically reconfigurable to define a penetrating end integrally formed with the elongated member.

### CROSS-REFERENCE TO RELATED APPLICATIONS

This application claims the benefit of U.S. Provisional Application No. 61/044,968, filed on Apr. 15, 2008.

### BACKGROUND

1. Technical Field

The present disclosure relates to an apparatus for the joining of tissue in surgical applications, and to methods for making the same. More particularly, the present disclosure relates to a surgical suturing apparatus for use during incision or wound repair, and methods of making the same.

2. Background of the Related Art

The structures and methods facilitating the attachment of a suture, or ligament, to a needle are well known in the art. Such needle-suture combinations are provided for a wide variety of monofilament and multifilament suture materials, in both the absorbable and non-absorbable varieties. These suture materials may be formed, for example, from catgut, silk, nylon, polyesters, polypropylene, steel, or absorbable synthetic materials such as polymers and copolymers of glycolic acid, lactic acid, dioxanone, caprolactone, and trimethylene carbonate.

Needle-suture combinations fall into two general classes, i.e. standard needle attachment, in which the suture is securely attached to the needle and is not intended to be separable therefrom except by cutting or severing, and removable or detachable needle attachment, in which the needle is separable from the suture in response to a force exerted by the clinician.

Various methods for both standard and detachable needle attachment are known in the art, one of the most conventional being the coupling of the shank end of a needle with the suture. However, when coupling a needle and suture in this manner, the possibility of inadvertent detachment of the needle from the suture exists. To address this potentiality, methods of integrally or monolithically forming the needle with the suture have been developed.

U.S. Pat. Nos. 5,531,761; 7,056,331; and 5,342,376 each relate to the use of a suture having a body that is integrally formed with a sharpened distal end, and suggest the inclusion of a material that is sufficiently rigid to facilitate the penetration of tissue with the distal end, such as a polymeric or co-polymeric materials.

U.S. Pat. No. 4,602,636 teaches the use of a wire suture formed of stainless steel or cobalt chromium alloys, for example, that includes a work hardened needle-like tip that is harder and stronger than the remainder of the suture.

Each of the methods for integrally forming a needle with a suture discussed above has associated disadvantages and difficulties which may be encountered during use or production. Accordingly, a need exists in the art for an improved surgical suturing apparatus, and a method of making the same, which overcomes these deficiencies.

### SUMMARY

In one aspect of the present disclosure, an apparatus for the joining of tissue is disclosed that includes an elongated member and a rigidifying agent. The rigidifying agent is associated with a distal end portion of the elongated member and increases the rigidity thereof in order to render the distal end portion mechanically reconfigurable such that a penetrating end integrally formed with the elongated member can be defined. In one embodiment, the penetrating end is configured to facilitate insertion of the apparatus into tissue.

In certain embodiments, the elongated member is formed of a biocompatible material, which may be bioabsorbable. In one embodiment, the elongated member is composed of a plurality of filaments arranged so as to define a plurality of interstices therebetween. In these embodiments, the rigidifying agent is at least partially disposed within the interstices of the distal end portion such that the rigidifying agent is maintained in the distal end portion in an amount substantially within the range of approximately 1% of the weight of the elongated member to approximately 150% of the weight of the elongated member. For example, the rigidifying agent may be maintained in the distal end portion at up to 20% of the weight of the elongated member. The rigidifying agent may be any biocompatible thermoplastic polymer, including but not limited to isocyanates, cyanoacrylates, cyanoacrylate monomers, photo polymerizable monomers, thermo polymerizable monomers, gamma-radiation polymerizable monomers, e.g., ultraviolet polymerizable monomers, and chemical polymerizable monomers.

The present disclosure contemplates that the elongated member may include a plurality of barbs. Alternatively, or additionally, the distal end portion of the elongated member may define a sharp tip, and in some embodiments, may be configured as a needle having, for example, an arcuate configuration or a distally tapered configuration.

The elongated member may include a weakened portion located proximally of the distal end portion to facilitate selective detachment thereof.

In another aspect of the present disclosure, a method of manufacturing an apparatus for the joining of tissue is disclosed. The method includes the steps of providing an elongated member formed of a biocompatible material, associating a rigidifying agent with a distal end portion thereof, increasing the rigidity of the rigidifying agent to thereby increase the rigidity of the distal end portion, and mechanically reconfiguring the distal end portion to define a penetrating end that is integrally formed with the elongated member.

The step of providing an elongated member may include providing an elongated material that is formed of a plurality of filaments defining interstices therebetween for retaining the rigidifying agent, in which case the step of associating the rigidifying agent with the distal end portion may include impregnation of the distal end portion with the rigidifying agent to thereby dispose the rigidifying agent within the interstices of the distal end portion.

The step of mechanically reconfiguring the distal end portion may include subjecting the distal end portion to heat and pressure to thereby form the aforementioned penetrating end.

The step of associating a rigidifying agent with the distal end portion may include the introduction of a biocompatible thermoplastic polymer. Examples of suitable biocompatible thermoplastic polymers include, but are not limited to cyanoacrylate monomers, PMMA (polymethyl methacrylate), PLGA (polylactic-co-glycolic acid), and polyhydroxyacetic acid.

These and other features of the apparatus disclosed herein, and methods of making the same, will become more readily apparent to those skilled in the art from the following detailed description of various embodiments of the present disclosure.

### BRIEF DESCRIPTION OF THE DRAWINGS

Various embodiments of the present disclosure are described hereinbelow with references to the drawings, wherein:

FIG. 1 is a side, perspective view of an exemplary apparatus for the joining of tissue;

FIG. 2 is a side, perspective view of one embodiment of the apparatus seen in FIG. 1 including a plurality of barbs formed on a distal portion thereof;

FIG. 3 is a side, perspective view of another embodiment of the apparatus seen in FIG. 1 including a weakened portion;

FIG. 4 is a side, perspective view of yet another embodiment of the apparatus seen in FIG. 1, in which the apparatus includes an elongated member comprised of a plurality of fibers;

FIG. 5 is a side, perspective view of an alternate embodiment of the apparatus shown in FIG. 4, in which the plurality of fibers are configured in a braid;

FIG. 6 is an enlarged view of the area of detail indicated in FIG. 5;

FIG. 7 is a top, perspective view of a mold comprising first and second mold portions, shown in spaced apart relation, for use in a method of manufacturing the apparatus seen in FIG. 1; and

FIG. 8 is a top, perspective view of the first and second mold portions seen in FIG. 7 shown in juxtaposed arrangement.

### DETAILED DESCRIPTION OF VARIOUS EMBODIMENTS

In the drawings, and in the description which follows, in which like references characters identify similar or identical elements, the term “proximal” should be understood as referring to the end of the apparatus that is closest to the clinician during use, whereas the term “distal” should be understood as referring to the end of the apparatus that is furthest from the clinician during use. In addition, use of the term “tissue” herein should be understood as referring to any bodily tissue including, but not limited to, skin, fascia, ligaments, tendons, muscle, and bone.

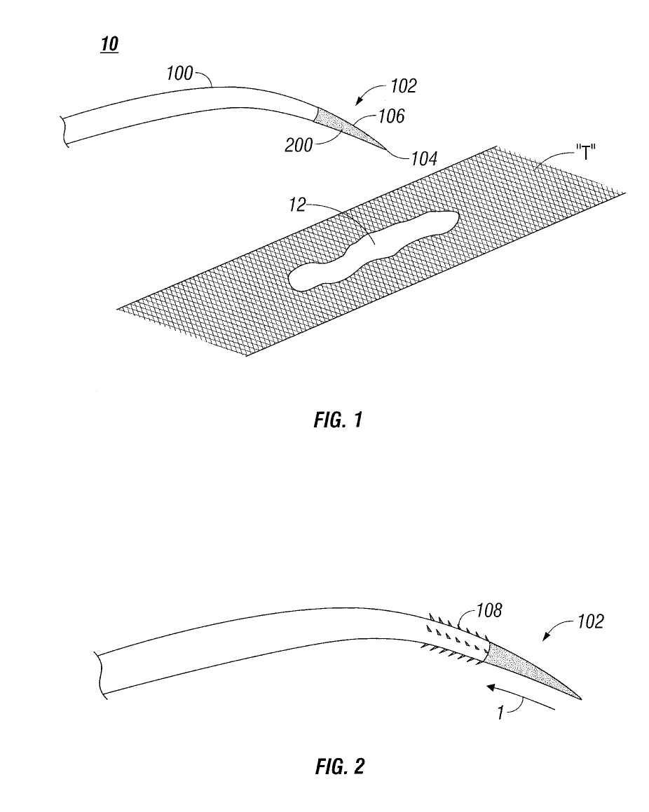

With reference now to FIG. 1, an apparatus 10 in accordance with the principles of the present disclosure is illustrated. The apparatus 10 includes an elongated member 100 having a distal end portion 102 that includes a penetrating end 104 formed integrally therewith. The penetrating end 104 may define a sharp tip, as seen in FIG. 1, for example, thereby enabling the apparatus 10 to penetrate tissue. In some embodiments, the penetrating end 104 may be configured and dimensioned so as to define a needle, or configured similarly in shape to a traditional needle, e.g., a steel needle.

Desirably, the elongated member 100 has a measure of flexibility such that the suturing apparatus 10 can be manipulated by the clinician to join adjacent sections of the tissue “T” together. As an illustrative example, the apparatus 10 may be employed to repair or close an incision 12, wound, or the like formed in the tissue “T” using conventional suturing techniques. The elongated member 100 may be any elongated member, e.g. a suture, ligature, or surgical tape, formed from a suitable biocompatible, including but not limited to polypropylene, polyester, nylon, or other polymeric materials. In one embodiment, it is envisioned that the elongated member 100 may be formed of a bioabsorbable material.

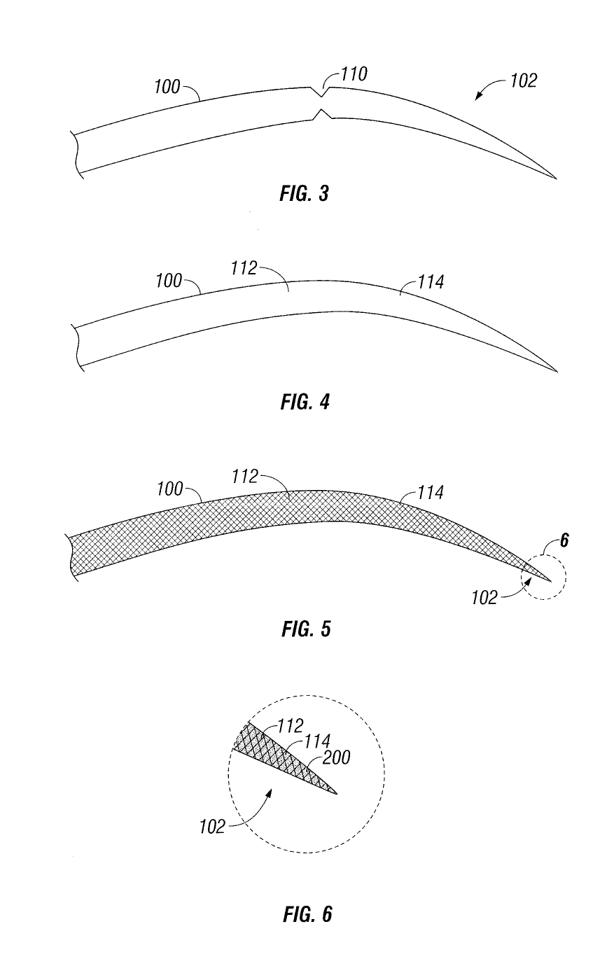

The distal end portion 102, and the penetrating end 104 formed integrally therewith, may exhibit any configuration that facilitates the penetration of tissue. Accordingly, the penetrating end 104 may be substantially incisive, as shown, or substantially blunt. As seen in FIG. 1, in one embodiment, the distal end portion 102 may be configured as a needle 106. The needle 106 may exhibit any configuration suitable for the intended purpose of facilitating the passage of the elongated member 100 through the tissue “T.” To this end, the needle 106 may define an arcuate or linear configuration, and may be tapered such that the surface area thereof decreases distally along its length. As seen in FIG. 2, the distal end portion 102 may include a plurality of barbs 108 formed thereon to inhibit removal, or reversal, of the distal end portion 102 from the tissue “T” (FIG. 1) in the proximal direction indicated by arrow 1. Additionally, or alternatively, the elongated member 100 may include a weakened portion 110 that is located proximally of the distal end portion 102, as seen in FIG. 3, such that the clinician may selectively detach the distal end portion 102 from the remainder of the elongated member 100 upon successfully joining the tissue “T” (FIG. 1).

Referring again to FIG. 1, prior to formation of the penetrating end 104, a rigidifying agent 200 is associated with the distal end portion 102. In one embodiment of the present disclosure, the rigidifying agent is a thermoplastic polymer, such as a cyanoacrylate monomer that would polymerize once it has penetrated into or coated the distal end portion 102. However, the use of other polymers, including but not limited to isocyanates, cyanoacrylates, cyanoacrylate monomers, photo polymerizable monomers, thermo polymerizable monomers, radiation polymerizable monomers, e.g., ultraviolet polymerizable monomers, and chemical polymerizable monomers, or polymerizations, e.g., photo-initiated polymerization, is not beyond the scope of the present disclosure.

The rigidifying agent 200 is adapted to transition from a first, or initial condition, to a second condition upon the application of energy thereto. The energy may be created in any suitable manner, and may be in the form of pressure, heat, or irradiation. Alternatively, the requisite energy may be created using a chemical reaction, e.g., curing. In the initial condition, the rigidifying agent 200 is substantially pliable and/or malleable such that it may be applied to the distal end portion 102. The rigidifying agent 200 may be applied to the distal end portion 102 in any suitable manner, such as by spraying or dip coating the distal end portion 102. In the second condition, the rigidifying agent 200 is substantially more rigid, thereby rendering the distal end portion 102 substantially more rigid as well and susceptible to mechanical reconfiguration to thereby define the penetrating end 104, as discussed in further detail below.

With reference to FIGS. 4-6, in one embodiment, the elongated member 100 is composed of a plurality of filaments 112. The filaments 112 are arranged to define a plurality of interstices 114 therebetween, and may be arranged in any manner suitable for this intended purpose, including but not limited to braiding, entangling, weaving, or comingling the plurality of filaments 112. The filaments 112 may be loosely interwoven, as seen in FIG. 4, or alternatively, the filaments may be arranged in a braided configuration, as seen in FIG. 5. In the embodiment of FIGS. 4-6, upon the association of the rigidifying agent 200 with the elongated member 100, at least a portion of the rigidifying agent 200 is disposed within the interstices of the distal end portion 102. The rigidifying agent may be maintained within the distal end portion in an amount substantially within the range of approximately 1% of the weight of the elongated member 100 to approximately 150% of the weight of the elongated member 100.

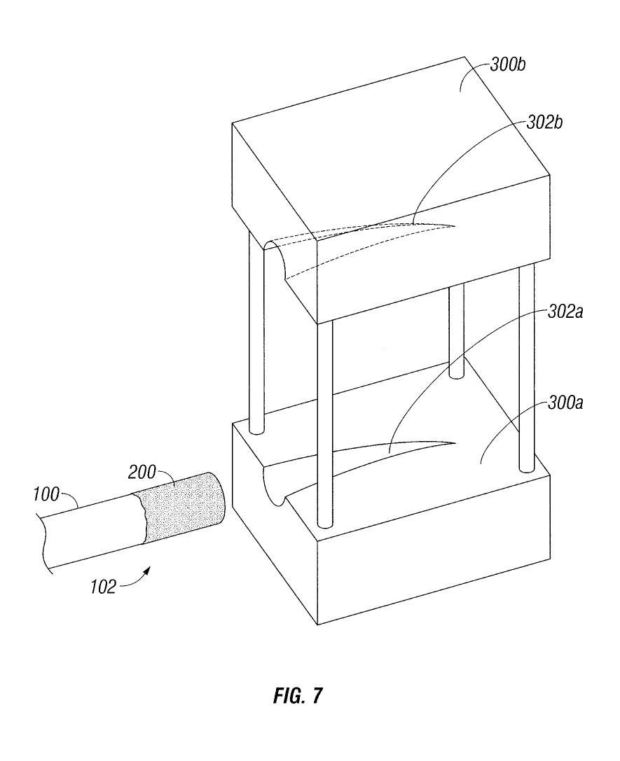

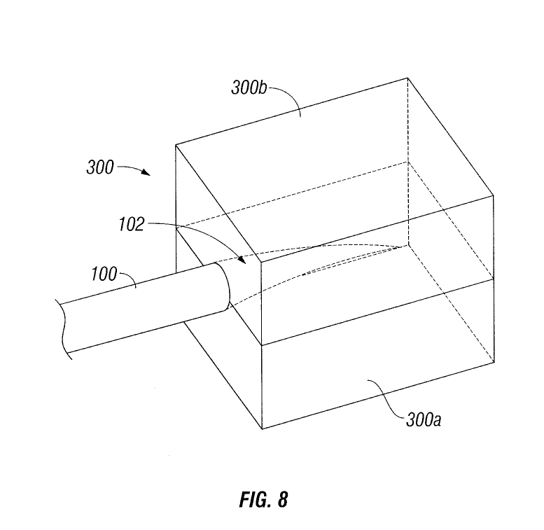

Referring now to FIGS. 1, 7, and 8, a method of manufacturing the apparatus 10 discussed above will be described. Initially, the elongated member 100 is provided and the rigidifying agent 200 is associated with the distal end portion 102 thereof. It should be noted that the elongated member 100 illustrated in FIG. 7 does not yet include the penetrating end 104 depicted in FIG. 1, as the penetrating end 104 is created during the process described below.

As previously discussed, the rigidifying agent 200 is in a substantially pliable and/or malleable condition during application to the distal portion 102 of the elongated member 100. Subsequently, however, the rigidifying agent 200 is caused to transition to the second condition, during which the rigidifying agent 200 experiences a substantial increase in rigidity concomitantly with the distal end portion 102. Upon realizing sufficient rigidity, the distal end portion 102 is mechanically reconfigured to define the penetrating end 104.

Referring still to FIGS. 1, 7, and 8, in one embodiment of the aforedescribed method, the rigidifying agent 200, and consequently, the distal end portion 102, are caused to rigidify through the application of heat and/or pressure thereto. One suitable manner in which the necessary heat and/or pressure may be created and applied is through the employ of compression molding. During this process, subsequent to the application of the rigidifying agent 200 to the distal end portion 102, the distal end portion 102 is placed within an open first mold portion 300_{A} (FIG. 7) having a first cavity 302_{A} formed therein that defines a configuration corresponding to that which is desired for the penetrating end 104 (FIG. 1) and the remainder of the distal end portion 102. Thereafter, a second mold portion 300_{B} with a second cavity 302_{B} formed therein is brought into juxtaposed arrangement with the first mold portion 300_{A}, as seen in FIG. 8, thereby applying a controllable level of pressure and/or heat to the distal end portion 102. The malleability of the rigidifying agent 200 in its first condition allows the distal end portion 102 to be reconfigured, e.g. reshaped, such that that distal end portion 102 exhibits the configuration collectively defined by the respective first and second cavities 302_{A}, 302_{B} of the first and second mold portions 300_{A}, 300_{B}. The pressure and/or heat applied to the distal end portion 102 transitions the rigidifying agent 200 from its first condition to its second condition, which facilitates the creation of an elongated member 100 having a substantially rigid penetrating end 104 formed integrally therewith.

In general, the second cavity 302_{B} will define a configuration that is substantially similar to that of the first cavity 302_{A}, although a mold portion 300 including respective first and second mold cavities 302_{A}, 302_{B} that are dissimilar is not beyond the scope of the present disclosure. It should be noted that the respective first and second cavities 302_{A}, 302_{B} may be configured to yield an elongated member 100 having a distal end portion 102 with an arcuate, tapered configuration, as discussed above with respect to FIG. 1, or a configuration that includes a plurality of barbs 108, as discussed above with respect to FIG. 2. Other methods which may be used to reconfigure the distal end portion 102 to define the penetrating end 104 are also within the purview of those skilled in the art, and include, but are not limited to the use of ultrasonic energy, blades, molds, and dies.

During the compression molding process, the mold portion 300 may be heated either prior, or subsequent, to the juxtaposition of the respective first and second mold portions 300_{A}, 300_{B}, such that a controllable level of heat may be applied to the distal end portion 102. The application of heat may act to further facilitate the reconfiguration of the distal end portion 102 and/or the transition of the rigidifying agent 200 from the first condition to the second condition.

For the purposes of discussion, in one embodiment, it is contemplated that the elongated member 100 may be a Polysorb™ multifilament absorbable suture that is treated with octyl cyanoacrylate as the rigidifying agent 200. The distal end portion 102 of the Polysorb™ suture is dipped into the octyl cyanoacrylate such that the octyl cyanoacrylate is disposed within the interstices 114 (FIG. 3) defined between the plurality of filaments 112 of the Polysorb™ suture and embedded within the distal end portion 102 thereof. The octyl cyanoacrylate is then allowed to cure, during which time the rigidity of the distal end portion 102 increases. Either during the curing process, or subsequently thereafter, the distal end portion 102 is placed into a mold, e.g., between the respective first and second mold portions 302_{A}, 302_{B} (FIGS. 7, 8), to form the penetrating end 104 into a needle-like shape, for example.

Although the method of manufacture disclosed herein and illustrated in FIGS. 7 and 8 has been discussed with respect to the elongated member 100 of the apparatus 10 (FIG. 1), in alternative embodiments of the present disclosure, it is envisioned that the method of manufacture may be employed in connection with various other structures. For example, the presently disclosed method of manufacture may be used in the fabrication of a self-gripping surgical mesh, such as the Parietex ProGrip™, which is distributed commercially by Covidien, 15 Hampshire Street, Mansfield, Mass., USA, for use during open inguinal hernia repair, and discussed in U.S. patent application Ser. No. 12/032,750, filed on Feb. 18, 2008, the entire contents of which are incorporated by reference herein.

The above description, disclosure, and figures should not be construed as limiting, but merely as exemplary of particular embodiments. It is to be understood, therefore, that the disclosure is not limited to the precise embodiments described, and that various other changes and modifications may be effected by one skilled in the art without departing from the scope or spirit of the disclosure. Additionally, those skilled in the art will appreciate that the elements and features illustrated or described in connection with one embodiment can be combined with those of another, and that such modifications and variations are also intended to be included within the scope of the present disclosure.

What is claimed is:

1. An apparatus for the joining of tissue, comprising:
an elongated member formed of a biocompatible material; and
a rigidifying agent integrated into a first end portion of the elongated member, wherein the rigidifying agent increases the rigidity of the first end portion such that the first end portion such that the first end portion defines a penetrating end integrally formed with the elongated member.

2. The apparatus of claim 1, wherein the penetrating end is configured to facilitate insertion of the apparatus into tissue.

3. The apparatus of claim 1, wherein the elongated member is formed of a bioabsorbable material.

4. The apparatus of claim 1, wherein the elongated member comprises a plurality of filaments.

5. The apparatus of claim 4, wherein the plurality of filaments are arranged so as to define interstices therebetween, the rigidifying agent being at least partially disposed within the interstices of the first end portion.

6. The apparatus of claim 1, wherein the rigidifying agent is maintained in the first end portion in an amount substantially within the range of approximately 1% of a weight of the elongated member to approximately 150% of the weight of the elongated member.

7. The apparatus of claim 6, wherein the rigidifying agent is maintained in the first end portion at up to 20% of the weight of the elongated member.

8. The apparatus of claim 1, wherein the elongated member includes a plurality of barbs.

9. The apparatus of claim 1, wherein the elongated member includes a weakened portion located proximally of the first end portion to facilitate selective detachment thereof.

10. The apparatus of claim 1, wherein the penetrating end defines a sharp tip.

11. The apparatus of claim 1, wherein the first end portion is configured as a needle.

12. The apparatus of claim 1, wherein the rigidifying agent is a biocompatible thermoplastic polymer.

13. The apparatus of claim 12, wherein the rigidifying agent is selected from the group consisting of cyanoacrylate monomers, isocyanates, silicones, and ultraviolet polymerizable polyacrylates.

14. An apparatus for the joining of tissue, comprising:
an elongated member formed of a biocompatible material; and
a rigidifying agent associated with a distal end portion of the elongated member, wherein the rigidifying agent increases the rigidity of the distal end portion such that the distal end portion is mechanically reconfigurable to define a penetrating end integrally formed with the elongated member.

15. A method of manufacturing an apparatus for the joining of tissue, comprising the steps of:
providing an elongated member formed of a biocompatible material;
associating a rigidifying agent with a distal end portion of the elongated member;
increasing the rigidity of the rigidifying agent to thereby increase the rigidity of the distal end portion; and
mechanically reconfiguring the distal end portion to define a penetrating end integrally formed with the elongated member.

16. The method of claim 15, wherein the step of providing an elongated member includes providing an elongated material formed of a plurality of filaments defining interstices therebetween for retaining the rigidifying agent.

17. The method of claim 16, wherein the step of associating the rigidifying agent with the distal end portion includes impregnating the distal end portion with the rigidifying agent to thereby dispose the rigidifying agent within the interstices of the distal end portion.

18. The method of claim 15, wherein the step of mechanically reconfiguring the distal end portion includes subjecting the distal end portion to heat and pressure to thereby form the penetrating end.

19. The method of claim 15, wherein the step of associating a rigidifying agent with the distal end portion includes associating a biocompatible thermoplastic polymer with the distal end portion.

20. The method of claim 19, wherein the step of associating a rigidifying agent with the distal end portion includes associating a polymer with the distal end portion that is selected from the group consisting of cyanoacrylate monomers, isocyanates, silicones, and ultraviolet polymerizable polyacrylates.
